(19) United States
(12) Patent Application Publication
(10) Pub. No.: US 2026/0363812 A1
(43) Pub. Date: Nov. 19, 2026

(54) Portal-Establishing Devices and Methods Thereof for Establishing Portals in Procedural Barriers

(71) Applicant: Bard Access Systems, Inc., Salt Lake City, UT (US)

(72) Inventors: Steffan Sowards, Salt Lake City, UT (US); Anthony K. Misener, Bountiful, UT (US)

(21) Appl. No.: 19/753,273

(22) Filed: Jul. 24, 2026

Related U.S. Application Data
(62) Division of application No. 17/499,688, filed on Oct. 12, 2021, now Pat. No. 12,690,935.
(60) Provisional application No. 63/091,185, filed on Oct. 13, 2020.

(51) Int. Cl. A61B 46/10; A61B 46/00; A61B 46/20

(52) U.S. Cl. CPC A61B 46/10; A61B 46/20; A61B 46/40

(57) ABSTRACT

Portal-establishing devices and methods thereof for establishing portals in procedural barriers. A portal-establishing device can include a portal and a breaching mechanism. The portal includes a portal barrier in a protruding-end portion thereof. The portal can be configured to enable one or more functional connections such as an optical connection or an electrical connection across a procedural barrier between a single-use medical device on a clinician-facing side of the procedural barrier and another medical device on a patient-facing side of the procedural barrier. The breaching mechanism can be configured to breach the procedural barrier and place the portal over a breach in the procedural barrier. Methods of the portal-establishing devices include methods of using the portal-establishing devices.

### PRIORITY

This application is a division of U.S. patent application Ser. No. 17/499,688, filed Oct. 12, 2021, now U.S. Pat. No. 12,690,935, which claims the benefit of priority to U.S. Provisional Application No. 63/091,185, filed Oct. 13, 2020, each of which is incorporated by reference in its entirety into this application.

### BACKGROUND

Procedural fields are typically established about patients with one or more procedural barriers before medical procedures. For example, sterile fields can be established over or around patients by covering the patients with sterile drapes. Oftentimes, the medical procedures require multiple-use medical devices that cannot be sterilized, which mandate placement of such medical devices under the sterile drapes; however, sterile single-use medical devices often need to be functionally connected to the multiple-use medical devices. Some existing solutions rely on breaching sterile drapes to make functional connections between sterile single-use medical devices and multiple-use medical devices. But arbitrarily breaching such sterile drapes risks contaminating the sterile fields carefully established about the patients. That, and establishing some functional connections such as optical connections between the sterile single-use medical devices and the multiple-use medical devices can be difficult, particularly through arbitrary, self-made breaches in the sterile drapes. What is needed are procedural barriers with medical device-connecting portals that facilitate establishing safe, routine, functional connections between sterile single-use medical devices and other medical devices such as multiple-use medical devices.

Disclosed herein are portal-establishing devices and methods thereof for establishing portals in procedural barriers.

### SUMMARY

Disclosed herein is a portal-establishing device including, in some embodiments, a portal and a breaching mechanism. The portal includes a portal barrier in a protruding-end portion thereof. The portal is configured to enable one or more functional connections across a procedural barrier between a single-use medical device on a clinician-facing side of the procedural barrier and another medical device on a patient-facing side of the procedural barrier. The breaching mechanism is configured to breach the procedural barrier and place the portal over a breach in the procedural barrier.

In some embodiments, the portal barrier is a transparent sheet of polymeric material. The sheet has sufficient optical transmissibility for establishing an optical connection across the sheet for the one-or-more functional connections between the single-use medical device and the other medical device.

In some embodiments, the sheet is sufficiently tear resistant for establishing an electrical connection across the sheet for the one-or-more functional connections between the single-use medical device and the other medical device without propagating any hole or tear made in the sheet of polymeric material to effectuate the electrical connection.

In some embodiments, the portal barrier is a split septum for establishing an optical connection, an electrical connection, or both the optical connection and the electrical connection across the septum for the one-or-more functional connections between the single-use medical device and the other medical device.

In some embodiments, the breaching mechanism includes an extension of the portal having internal threads configured to screw together with a protruding portion of the other medical device having external threads. The extension of the portal is configured to breach the procedural barrier with an internal cutting edge when screwed together with the protruding portion of the other medical device with the procedural barrier therebetween.

In some embodiments, the breaching mechanism includes an extension of the portal having one or more internal magnetic elements configured to couple with one or more external magnetic elements of a protruding portion of the other medical device. The extension of the portal is configured to breach the procedural barrier with an internal cutting edge when coupled together with the protruding portion of the other medical device with the procedural barrier therebetween.

In some embodiments, the breaching mechanism includes an extension of the portal having external threads configured to screw together with a protruding portion of the other medical device having internal threads. The extension of the portal is configured to breach the procedural barrier with an external cutting edge when screwed together with the protruding portion of the other medical device with the procedural barrier therebetween.

In some embodiments, the breaching mechanism includes a cap coupled by a hinge pin to a rigid band configured to sit over a protruding portion of the other medical device with the procedural barrier therebetween. The cap is configured to breach the procedural barrier with a cutting edge when closed over the band.

In some embodiments, the portal extends from the cap along a centerline of the cap such that the portal barrier is aligned with the breach in the procedural barrier when the procedural barrier is breached by the cap.

In some embodiments, the breaching mechanism includes a cap configured to sit over a protruding portion of the other medical device with the procedural barrier therebetween. An extension arm coupled by a hinge pin to the cap is configured to breach the procedural barrier with a punch of the extension arm when the punch is pushed through the procedural barrier by articulating the extension arm.

In some embodiments, the portal extends from the extension arm along a centerline of the punch but opposite thereof such that the portal barrier is aligned with the breach in the procedural barrier when the procedural barrier is breached by the punch.

In some embodiments, the breaching mechanism includes a rigid band configured to sit over a protruding portion of the other medical device with the procedural barrier therebetween. A cap coupled by a hinge pin to the band is configured to breach the procedural barrier with a punch of the cap when the punch is pushed through the procedural barrier with by way of articulating the cap.

In some embodiments, the portal extends from the cap along a centerline of the punch but opposite thereof such that the portal barrier is aligned with the breach in the procedural barrier when the procedural barrier is breached by the punch.

In some embodiments, the portal-establishing device is sterile and the procedural barrier is a sterile drape.

Also disclosed herein is a method of a portal-establishing device. The method includes a procedural barrier-breaching step and a portal-placing step. The procedural barrier-breaching step includes breaching a procedural barrier between a single-use medical device on a clinician-facing side of the procedural barrier and another medical device on a patient-facing side of the procedural barrier with a breaching mechanism of the portal-establishing device. The portal-placing step includes placing a portal over a breach in the procedural barrier. The portal-placing step enables one or more functional connections between the single-use medical device and the other medical device through the portal of the portal-establishing device.

In some embodiments, the method further includes a functional connection-establishing step. The functional connection-establishing step includes establishing an optical connection for the one-or-more functional connections across a transparent sheet of polymeric material having sufficient optical transmissibility for the optical connection as the portal barrier.

In some embodiments, the functional connection-establishing step includes establishing an electrical connection for the one-or-more functional connections across the sheet. The sheet has sufficient tear resistance for the functional connection-establishing step when establishing the electrical connection without propagating any hole or tear made in the sheet to effectuate the electrical connection.

In some embodiments, the method further includes a functional connection-establishing step. The functional connection-establishing step includes establishing an optical connection, an electrical connection, or both the optical connection and the electrical connection for the one-or-more functional connections across a split septum as the portal barrier.

In some embodiments, the method further includes a device-mating step. The device-mating step includes screwing together an extension of the portal having internal threads and a protruding portion of the other medical device having external threads. The procedural barrier-breaching step further includes breaching the procedural barrier between the extension of the portal and the protruding portion of the other medical device with an internal cutting edge of the extension of the portal while performing the device-mating step.

In some embodiments, the method further includes a device-mating step. The device-mating step includes magnetically coupling together an extension of the portal having one or more internal magnetic elements and a protruding portion of the other medical device having one or more external magnetic elements. The procedural barrier-breaching step further includes breaching the procedural barrier between the extension of the portal and the protruding portion of the other medical device with an internal cutting edge of the extension of the portal while performing the device-mating step.

In some embodiments, the method further includes a device-mating step. The device-mating step includes screwing together an extension of the portal having external threads and a protruding portion of the other medical device having internal threads. The procedural barrier-breaching step further includes breaching the procedural barrier between the extension of the portal and the protruding portion of the other medical device with an external cutting edge of the extension of the portal while performing the device-mating step.

In some embodiments, the procedural barrier-breaching step further includes closing a cap coupled by a hinge pin to a rigid band over a protruding portion of the other medical device. In addition, the procedural barrier-breaching step includes breaching the procedural barrier between the cap and the protruding portion of the other medical device with a cutting edge of the cap while closing the cap over the protruding portion of the other medical device.

In some embodiments, the procedural barrier-breaching step further includes articulating an extension arm coupled by a hinge pin to a cap over a protruding portion of the other medical device. In addition, the procedural barrier-breaching step includes breaching the procedural barrier between the cap and the protruding portion of the other medical device by pushing a punch of the extension arm through the procedural barrier while articulating the extension arm.

In some embodiments, the procedural barrier-breaching step further includes articulating a cap coupled by a hinge pin to a rigid band over a protruding portion of the other medical device. In addition, the procedural barrier-breaching step includes breaching the procedural barrier between the cap and the protruding portion of the other medical device by pushing a punch of the cap through the procedural barrier while articulating the cap.

In some embodiments, the portal-establishing device is sterile and the procedural barrier is a sterile drape.

Also disclosed herein is a procedural barrier including, in some embodiments, a portal integrated into the procedural barrier. The portal includes a portal barrier in a protruding-end portion thereof configured to enable one or more functional connections across the procedural barrier between a single-use medical device on a clinician-facing side of the procedural barrier and another medical device on a patient-facing side of the procedural barrier.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates functionally connected single-use and multiple-use medical devices of an optical shape-sensing system in accordance with some embodiments.

FIG. 2 illustrates a portal having a portal barrier of a transparent sheet of polymeric material in accordance with some embodiments.

FIG. 3 illustrates a portal having a portal barrier of a splitable septum in accordance with some embodiments.

FIG. 4 illustrates a first portal-establishing device having a first breaching mechanism in accordance with some embodiments.

FIG. 5 illustrates a second portal-establishing device having a second breaching mechanism in accordance with some embodiments.

FIG. 6 illustrates a third portal-establishing device having a third breaching mechanism in accordance with some embodiments.

FIG. 7 illustrates a fourth portal-establishing device having a fourth breaching mechanism in accordance with some embodiments.

FIG. 8 illustrates a fifth portal-establishing device having a fifth breaching mechanism in accordance with some embodiments.

FIG. 9 illustrates a sixth portal-establishing device having a sixth breaching mechanism in accordance with some embodiments.

FIG. 10 illustrates a seventh portal-establishing device having a seventh breaching mechanism in accordance with some embodiments.

FIG. 11 illustrates an eighth portal-establishing device having an eighth breaching mechanism in accordance with some embodiments.

FIG. 12 illustrates a ninth portal-establishing device having a ninth breaching mechanism in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

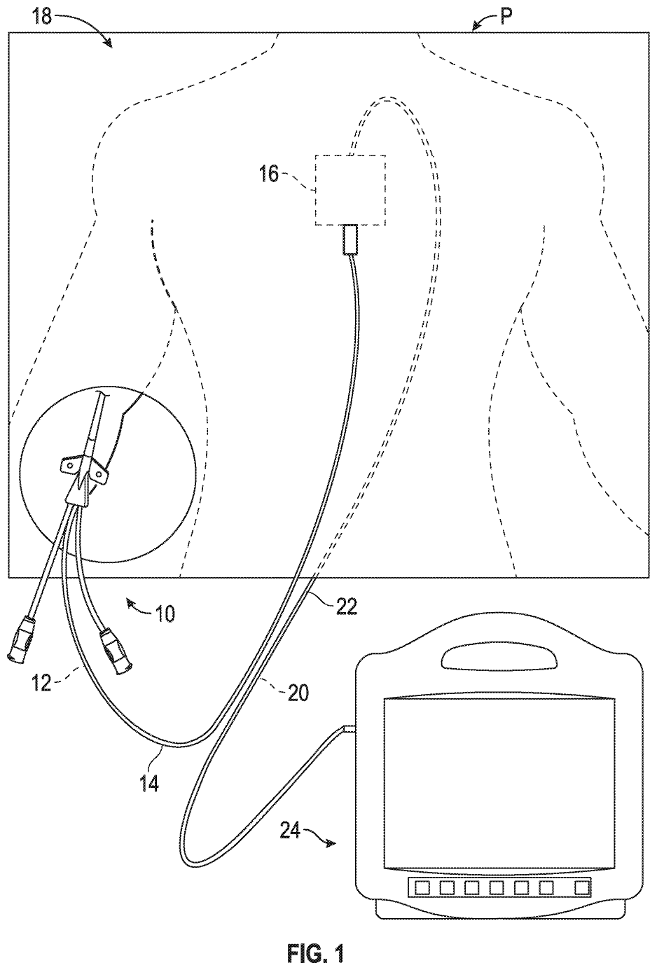

FIG. 1 illustrates functionally connected single-use and multiple-use medical devices of an optical shape-sensing system in accordance with some embodiments.

As set forth above, medical procedures often require multiple-use medical devices that cannot be sterilized, which mandate placement of such medical devices under sterile drapes; however, sterile single-use medical devices often need to be functionally connected to the multiple-use medical devices. Indeed, as shown, the optical shape-sensing system includes a single-use peripherally inserted central catheter (“PICC”) 10 having an optical-fiber stylet 12 disposed in an extension tube 14 functionally connected to a relay module 16 over a patient P but under a sterile drape 18 outside of a sterile field. The relay module 16, in turn, includes an optical fiber 20 disposed in a patch cable 22 functionally connected to a console 24 or an optical interrogator thereof that is also outside of the sterile field. Again, some existing solutions rely on breaching sterile drapes (e.g., the sterile drape 18) to make functional connections between sterile single-use medical devices (e.g., the PICC 10) and multiple-use medical devices (e.g., the relay module 16, the console 24, etc.). But arbitrarily breaching such sterile drapes risks contaminating the sterile fields carefully established about the patients. That, and establishing some functional connections such as optical connections between the sterile single-use medical devices and the multiple-use medical devices can be difficult, particularly through arbitrary, self-made breaches in the sterile drapes. What is needed are procedural barriers with medical device-connecting portals that facilitate establishing safe, routine, functional connections between sterile single-use medical devices and other medical devices such as multiple-use medical devices.

Disclosed herein are portal-establishing devices and methods thereof for establishing portals in procedural barriers.

### Portal-Establishing Devices

Each portal-establishing device of those disclosed herein includes a portal and a breaching mechanism. Description for portals is set forth immediately below. Description for the breaching mechanisms is set forth thereafter in the context of one or more of the portal-establishing devices.

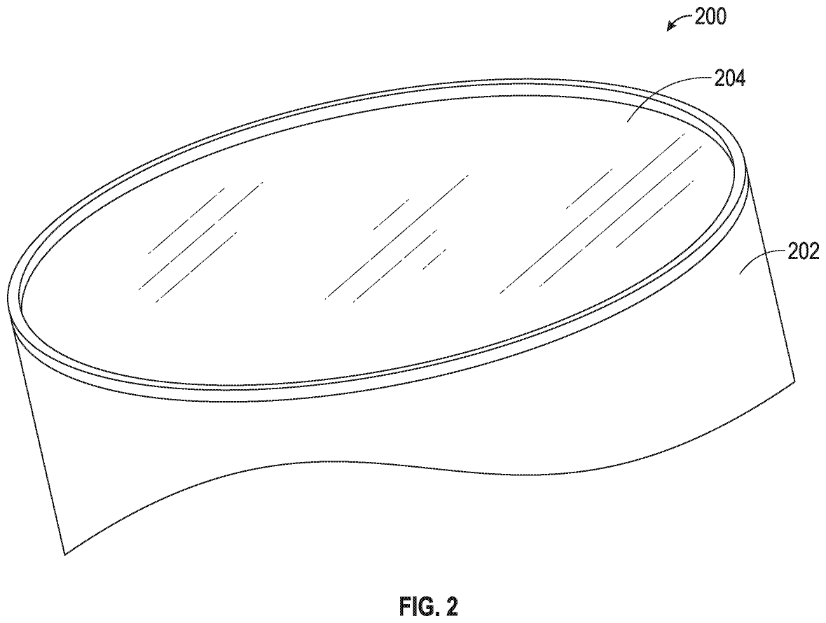

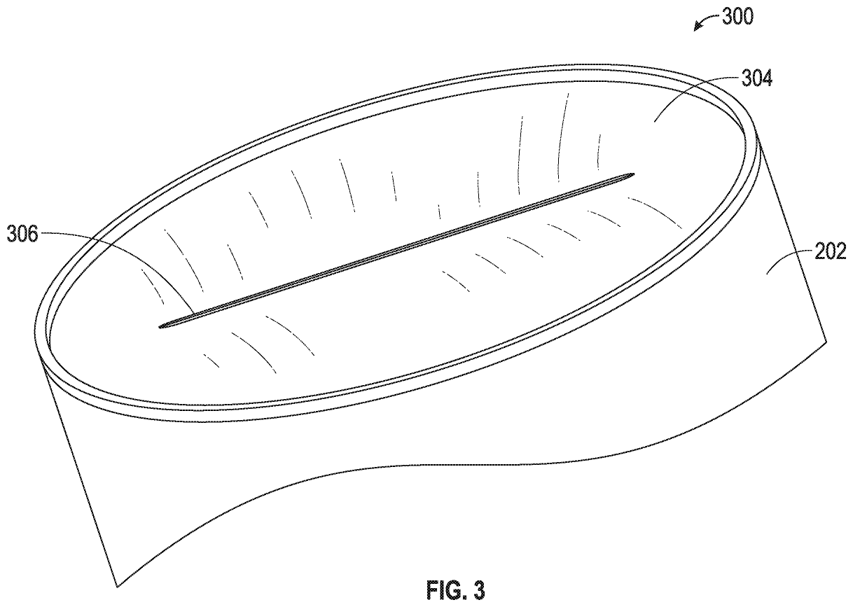

FIGS. 2 and 3 illustrate alternative portals 200 and 300 of the portal-establishing devices in accordance with some embodiments.

As shown, the portal 200 or 300 is a conduit 202 (e.g., a hollow right cylinder) including a portal barrier 204 or 304 in a protruding-end portion of the portal 200 or 300 or the conduit 202 thereof that protrudes from a portal-establishing device. The portal 200 or 300 is configured to enable one or more functional connections (e.g., an optical connection, an electrical connection, etc.) across a procedural barrier (e.g., the sterile barrier 18) between a single-use medical device (e.g., the PICC 10) on a clinician-facing side of the procedural barrier and another medical device such as a multiple-use medical device (e.g., the relay 16, the console 24, etc.) on a patient-facing side of the procedural barrier.

FIG. 2 illustrates the portal 200 having the portal barrier 204 of a transparent sheet of polymeric material in accordance with some embodiments.

The portal barrier 204 is a transparent sheet of polymeric material such as a single non-laminated sheet of polymeric material or two-or-more layers of polymeric material laminated together in a laminated sheet. The sheet can have sufficient optical transmissibility for establishing an optical connection across the sheet for the one-or-more functional connections between the single-use medical device and the multiple-use medical device. The sheet can be sufficiently tear resistant for establishing an electrical connection across the sheet for the one-or-more functional connections between the single-use medical device and the multiple-use medical device without propagating any hole or tear made in the sheet of polymeric material to effectuate the electrical connection. The polymeric material of the sheet can be a polyethylene, a polypropylene, or a polyurethane. For the laminated sheet, each layer of the two-or-more layers can include a same or different polymeric material than an adjacent layer of the two-or-more layers of the laminated sheet.

FIG. 3 illustrates the portal 300 having the portal barrier 304 of a splitable septum in accordance with some embodiments.

The portal barrier 304 is a split septum having a split 306 therethrough configured for establishing an optical connection, an electrical connection, or both the optical connection and the electrical connection across the septum for the one-or-more functional connections between the single-use medical device and the multiple-use medical device. When the conduit 202 of the portal 300 is formed of a flexible material (e.g., a thermoplastic such as polyethylene), opposite sides of the protruding-end portion of the portal 300 or the conduit 202 thereof can be squeezed together to open the septum for the one-or-more functional connections. Notably, the opposite sides of the protruding-end portion of the portal 300 or the conduit 202 thereof should be those at ends of the split 306 of the split septum. The polymeric material of the split septum can be a rubber such as a silicone rubber.

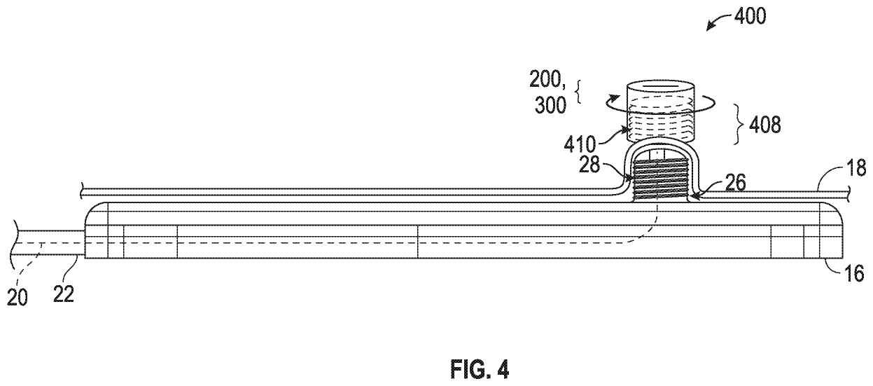

FIGS. 4-12 illustrate portal-establishing devices 400-1200 having different breaching mechanisms in accordance with some embodiments.

Each breaching mechanism of the portal-establishing devices 400-1200 set forth below is configured to breach the procedural barrier (e.g., the sterile barrier 18) and place the portal 200 or 300 over a breach in the procedural barrier. Notably, the breaching mechanism is oftentimes configured to breach the procedural barrier and place the portal 200 or 300 over the breach in the procedural barrier in one operation of the breaching mechanism, which operation can consist of a single motion in some embodiments. As set forth above, the portal 200 or 300 is configured to enable the one-or-more functional connections (e.g., an optical connection, an electrical connection, etc.) across the procedural barrier between the single-use medical device (e.g., the PICC 10) on the clinician-facing side of the procedural barrier and the multiple-use medical device (e.g., the relay 16, the console 24, etc.) on the patient-facing side of the procedural barrier.

FIG. 4 illustrates a first portal-establishing device 400 having a first breaching mechanism in accordance with some embodiments.

As shown, the first breaching mechanism includes an extension 408 of the portal 200 or 300 having internal threads 410 configured to screw together with a protruding portion 26 of the multiple-use medical device (e.g., the relay 16) having external threads 28. The extension 408 of the portal 200 or 300 is configured to breach the procedural barrier with an internal cutting edge (e.g., a sharpened section of the internal threads 410) when screwed together with the protruding portion 26 of the multiple-use medical device with the procedural barrier therebetween.

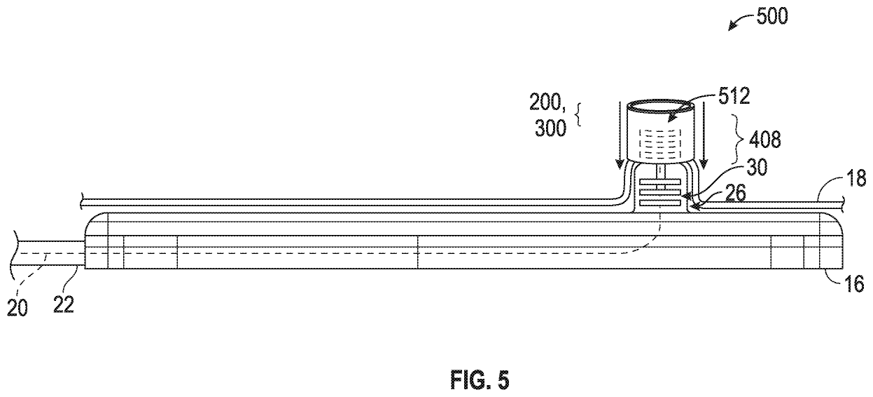

FIG. 5 illustrates a second portal-establishing device 500 having a second breaching mechanism in accordance with some embodiments.

As shown, the breaching mechanism includes the extension 408 of the portal 200 or 300 having one or more internal magnetic elements 512 configured to couple with one or more external magnetic elements 30 of the protruding portion 26 of the multiple-use medical device (e.g., the relay 16). The extension 408 of the portal 200 or 300 is configured to breach the procedural barrier with an internal cutting edge (not shown) when coupled together with the protruding portion 26 of the multiple-use medical device with the procedural barrier therebetween.

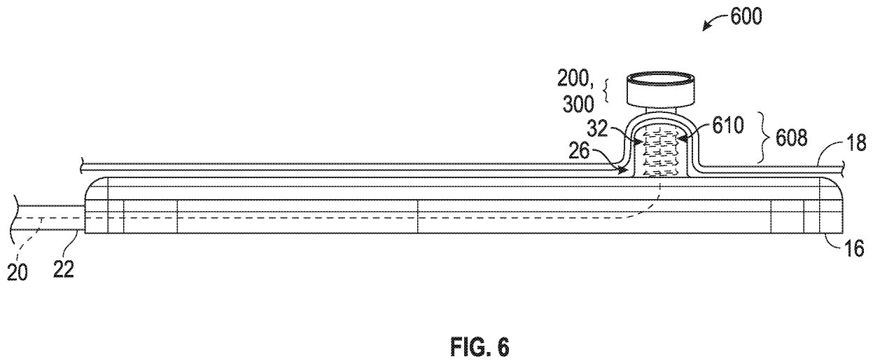

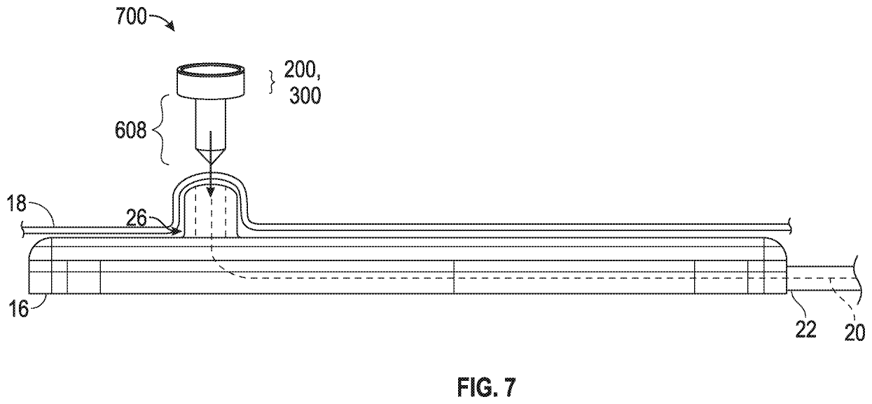

FIG. 6 illustrates a third portal-establishing device 600 having a third breaching mechanism in accordance with some embodiments, and FIG. 7 illustrates a fourth portal-establishing device 700 having a fourth breaching mechanism in accordance with some embodiments.

As shown in FIG. 6, the breaching mechanism includes an extension 608 of the portal 200 or 300 having external threads 610 configured to screw together with the protruding portion 26 of the multiple-use medical device (e.g., the relay 16) having internal threads 32. The extension 608 of the portal 200 or 300 is configured to breach the procedural barrier with an external cutting edge (e.g., a sharp tip of the extension 608, a sharpened section of the external threads 610, etc.) when screwed together with the protruding portion 26 of the multiple-use medical device with the procedural barrier therebetween.

As shown in FIG. 7, however, the breaching mechanism need not include the external threads 610 on the extension 608 of the portal 200 or 300. Indeed, the extension 608 of the portal 200 or 300 can be configured to breach the procedural barrier with a sharp tip of the extension 608 when inserted into the protruding portion 26 of the multiple-use medical device with the procedural barrier therebetween.

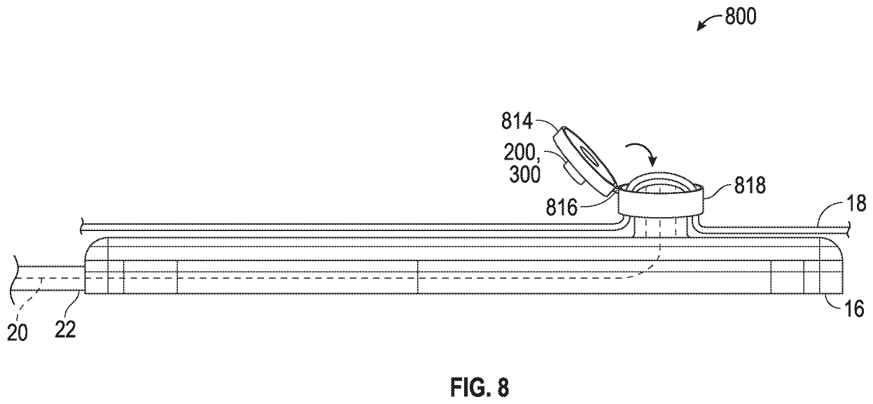

FIG. 8 illustrates a fifth portal-establishing device 800 having a fifth breaching mechanism in accordance with some embodiments.

As shown, the breaching mechanism includes a cap 814 coupled by a hinge pin 816 to a rigid band 818 configured to sit over the protruding portion 26 of the multiple-use medical device (e.g., the relay 16) with the procedural barrier therebetween. The cap 814 is configured to breach the procedural barrier with a cutting edge (e.g., an edge of the cap 814) when closed over the band 818. The portal extends 200 or 300 from the cap 814 along a centerline of the cap 814 such that the portal barrier 204 or 304 is aligned with the breach in the procedural barrier when the procedural barrier is breached by the cap 814.

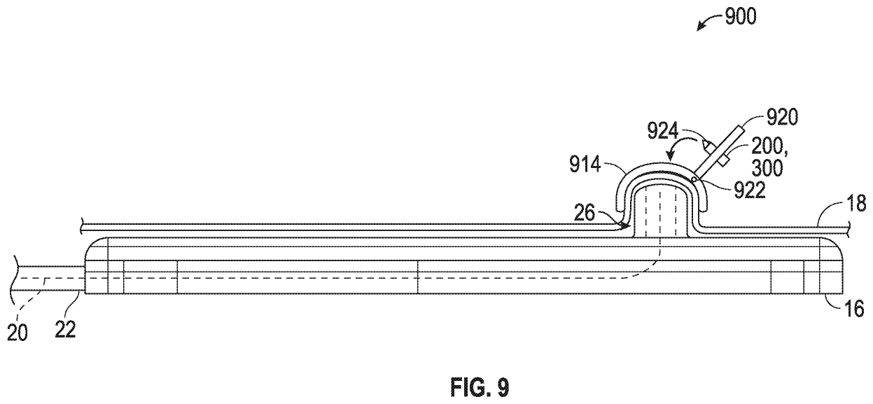

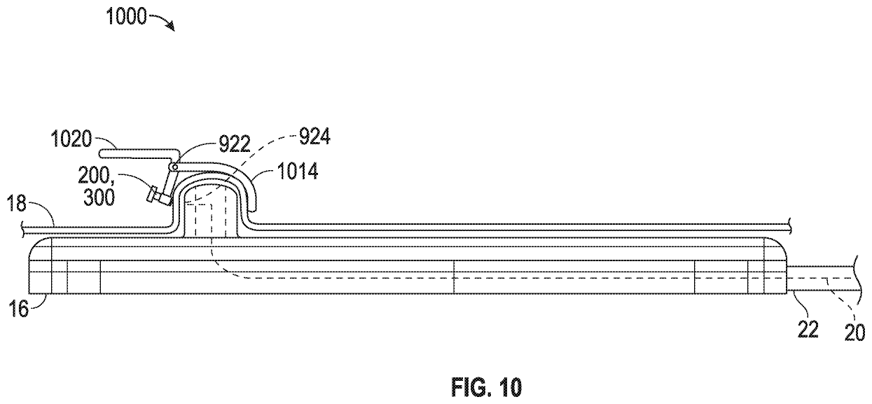

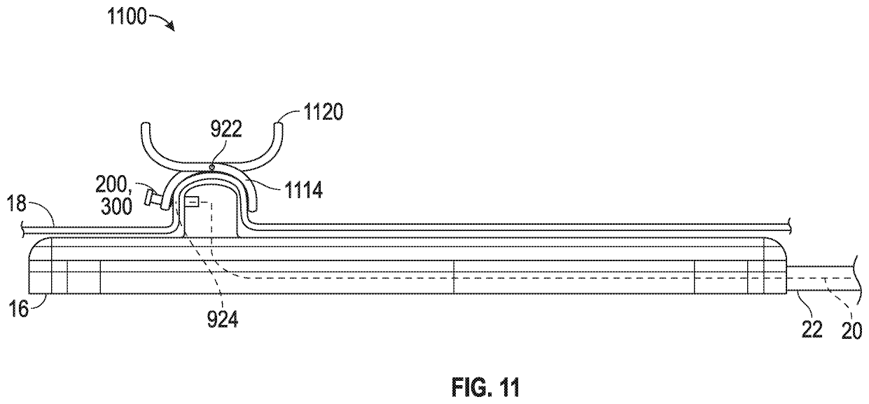

FIG. 9 illustrates a sixth portal-establishing device 900 having a sixth breaching mechanism in accordance with some embodiments. FIG. 10 illustrates a seventh portal-establishing 1000 device having a seventh breaching mechanism in accordance with some embodiments. FIG. 11 illustrates an eighth portal-establishing device 1100 having an eighth breaching mechanism in accordance with some embodiments.

As shown, the breaching mechanism includes a cap 914, 1014, or 1114 configured to sit over the protruding portion 26 of the multiple-use medical device (e.g., the relay 16) with the procedural barrier therebetween. An extension arm 920, 1020, or 1120 having a lever arm configured to complement a configuration of the cap 914, 1014, or 1114 is coupled by a hinge pin 922 to the cap 914, 1014, or 1114. The extension arm 920, 1020, or 1120 is configured to breach the procedural barrier with a punch 924 of the extension arm 920, 1020, or 1120 when the punch 924 is pushed through the procedural barrier by articulating the extension arm 920. The portal 200 or 300 extends from the extension arm 920, 1020, or 1120 along a centerline of the punch 924 but opposite thereof such that the portal barrier 204 or 304 is aligned with the breach in the procedural barrier when the procedural barrier is breached by the punch 924.

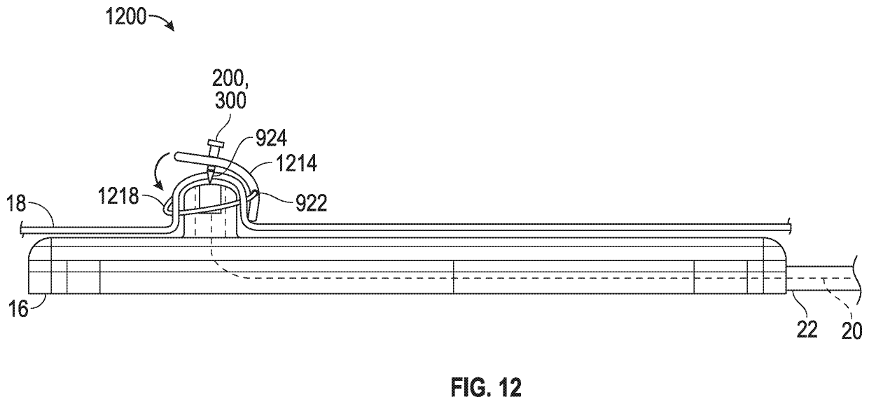

FIG. 12 illustrates a ninth portal-establishing device 1200 having a ninth breaching mechanism in accordance with some embodiments.

As shown, the breaching mechanism includes a rigid band 1218 configured to sit over the protruding portion 26 of the multiple-use medical device (e.g., the relay 16) with the procedural barrier therebetween. A cap 1214 coupled by the hinge pin 922 to the band 1218 is configured to breach the procedural barrier with the punch 924 of the cap 1214 when the punch 924 is pushed through the procedural barrier with by way of articulating the cap 1214. The portal 200 or 300 extends from the cap 1214 along a centerline of the punch 924 but opposite thereof such that the portal barrier 204 or 304 is aligned with the breach in the procedural barrier when the procedural barrier is breached by the punch 924.

### Procedural Barriers

While not shown, a procedural barrier (e.g., the sterile barrier 18) can include the portal 200 or 300 integrated into the procedural barrier. As set forth above, the portal 200 or 300 includes the portal barrier 204 or 304 in the protruding-end portion thereof configured to enable one or more functional connections across the procedural barrier 204 or 304 between a single-use medical device on a clinician-facing side of the procedural barrier and another medical device on a patient-facing side of the procedural barrier as a multiple-use medical device (e.g., the relay 16, the console 24, etc.) on a patient-facing side of the procedural barrier.

### Methods

Methods of the portal-establishing devices 400-1200 include methods of using the portal-establishing devices 400-1200. Each method of the methods of using the portal-establishing devices 400-1200 includes at least a procedural barrier-breaching step and a portal-placing step.

The procedural barrier-breaching step includes breaching a procedural barrier (e.g., the sterile barrier 18) between a single-use medical device (e.g., the PICC 10) on a clinician-facing side of the procedural barrier and another medical device such as a multiple-use medical device (e.g., the relay 16, the console 24, etc.) on a patient-facing side of the procedural barrier with a breaching mechanism of the portal-establishing device 400, 500, 600, 700, 800, 900, 1000, 1100, or 1200. Depending upon the portal-establishing device 400, 500, 600, 700, 800, 900, 1000, 1100, or 1200, the procedural barrier-breaching step includes one or more additional features or sub-steps as set forth below for each portal-establishing device of the foregoing portal-establishing devices.

The portal-placing step includes placing the portal 200 or 300 over a breach in the procedural barrier. The portal-placing step enables one or more functional connections between the single-use medical device and the multiple-use medical device through the portal 200 or 300 of the portal-establishing device 400, 500, 600, 700, 800, 900, 1000, 1100, or 1200.

FIG. 4 further illustrates a portion of the method of using the first portal-establishing device 400 in accordance with some embodiments.

As shown, the method further includes a device-mating step. The device-mating step includes screwing together the extension 408 of the portal 200 or 300 having the internal threads 410 and the protruding portion 26 of the multiple-use medical device (e.g., the relay 16) having the external threads 28. In accordance with the device-mating step, the procedural barrier-breaching step further includes breaching the procedural barrier (e.g., the sterile barrier 18) between the extension 408 of the portal 200 or 300 and the protruding portion 26 of the multiple-use medical device with the internal cutting edge of the extension 408 of the portal 200 or 300 while performing the device-mating step.

FIG. 5 further illustrates a portion of the method of using the second portal-establishing device 500 in accordance with some embodiments.

As shown, the method further includes a device-mating step. The device-mating step includes magnetically coupling together the extension 408 of the portal 200 or 300 having the one-or-more internal magnetic elements 512 and the protruding portion 26 of the multiple-use medical device (e.g., the relay 16) having the one-or-more external magnetic elements 30. In accordance with the device-mating step, the procedural barrier-breaching step further includes breaching the procedural barrier (e.g., the sterile barrier 18) between the extension 408 of the portal 200 or 300 and the protruding portion 26 of the multiple-use medical device with the internal cutting edge of the extension 408 of the portal 200 or 300 while performing the device-mating step.

FIG. 6 further illustrates a portion of the method of using the third portal-establishing device 600 in accordance with some embodiments.

As shown, the method further includes a device-mating step. The device-mating step includes screwing together the extension 608 of the portal 200 or 300 having the external threads 610 and the protruding portion 26 of the multiple-use medical device (e.g., the relay 16) having the internal threads 32. In accordance with the device-mating step, the procedural barrier-breaching step further includes breaching the procedural barrier (e.g., the sterile barrier 18) between the extension 608 of the portal 200 or 300 and the protruding portion 26 of the multiple-use medical device with the external cutting edge of the extension 608 of the portal 200 or 300 while performing the device-mating step.

FIG. 8 further illustrates a portion of the method of using the fifth portal-establishing device 800 in accordance with some embodiments.

As shown, the procedural barrier-breaching step further includes closing the cap 814 coupled by the hinge pin 816 to the rigid 818 band over the protruding portion 26 of the multiple-use medical device (e.g., the relay 16). In addition, the procedural barrier-breaching step includes breaching the procedural barrier (e.g., the sterile barrier 18) between the cap 814 and the protruding portion 26 of the multiple-use medical device with the cutting edge of the cap 814 while closing the cap 814 over the protruding portion 26 of the multiple-use medical device.

FIG. 9 further illustrates a portion of the method of using the sixth portal-establishing device 900 in accordance with some embodiments. FIG. 10 further illustrates a portion of the method of using the seventh portal-establishing 1000 device having a seventh breaching mechanism. FIG. 11 further illustrates a portion of the method of using the eighth portal-establishing device 1100 in accordance with some embodiments.

As shown, the procedural barrier-breaching step further includes articulating the extension arm 920, 1020, or 1120 coupled by the hinge pin 922 to the cap 914, 1014, or 1114 over the protruding portion 26 of the multiple-use medical device (e.g., the relay 16). In addition, the procedural barrier-breaching step includes breaching the procedural barrier (e.g., the sterile barrier 18) between the cap 914, 1014, or 1114 and the protruding portion 26 of the multiple-use medical device by pushing the punch 924 of the extension arm 920, 1020, or 1120 through the procedural barrier while articulating the extension arm 920, 1020, or 1120.

FIG. 12 further illustrates a portion of the method of using the ninth portal-establishing device 1200 in accordance with some embodiments.

As shown, the procedural barrier-breaching step further includes articulating the cap 1214 coupled by the hinge pin 922 to the rigid band 1218 over the protruding portion 26 of the multiple-use medical device (e.g., the relay 16). In addition, the procedural barrier-breaching step includes breaching the procedural barrier (e.g., the sterile barrier 18) between the cap 1214 and the protruding portion 26 of the multiple-use medical device by pushing the punch 924 of the cap 1214 through the procedural barrier while articulating the cap 1214.

Again, FIG. 2 illustrates the portal 200 having the portal barrier 204 of the transparent sheet of polymeric material in accordance with some embodiments.

The method can further include a functional connection-establishing step in view of the portal 200. The functional connection-establishing step can include establishing an optical connection for the one-or-more functional connections across the transparent sheet of polymeric material having sufficient optical transmissibility for the optical connection as the portal barrier (e.g., the portal barrier 204).

The functional connection-establishing step can additionally or alternatively include establishing an electrical connection for the one-or-more functional connections across the sheet. The sheet has sufficient tear resistance for the functional connection-establishing step when establishing the electrical connection without propagating any hole or tear made in the sheet to effectuate the electrical connection.

Again, FIG. 3 illustrates the portal 300 having the portal barrier 304 of the splitable septum in accordance with some embodiments.

The method can further include a functional connection-establishing step in view of the portal 300. The functional connection-establishing step can include establishing an optical connection, an electrical connection, or both the optical connection and the electrical connection for the one-or-more functional connections across a split septum as the portal barrier (e.g., the portal barrier 304).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A portal-establishing device, comprising:
a portal including a portal barrier in a protruding-end portion thereof, the portal configured to enable one or more functional connections across a procedural barrier between a single-use medical device on a clinician-facing side of the procedural barrier and a second medical device on a patient-facing side of the procedural barrier; and
a breaching mechanism configured to breach the procedural barrier and place the portal over a breach in the procedural barrier, wherein the breaching mechanism includes one or both of a cap and a rigid band configured to sit over a protruding portion of the second medical device with the procedural barrier therebetween.

2. The portal-establishing device of claim 1, wherein the cap is coupled by a hinge pin to the rigid band, the cap configured to breach the procedural barrier with a cutting edge when closed over the band.

3. The portal-establishing device of claim 2, wherein the portal extends from the cap along a centerline of the cap such that the portal barrier is aligned with the breach in the procedural barrier when the procedural barrier is breached by the cap.

4. The portal-establishing device of claim 1, wherein an extension arm is coupled by a hinge pin to the cap, the extension arm configured to breach the procedural barrier with a punch of the extension arm when the punch is pushed through the procedural barrier by articulating the extension arm.

5. The portal-establishing device of claim 4, wherein the portal extends from the extension arm along a centerline of the punch but opposite thereof such that the portal barrier is aligned with the breach in the procedural barrier when the procedural barrier is breached by the punch.

6. The portal-establishing device of claim 1, wherein the cap is coupled by a hinge pin to the band, the cap configured to breach the procedural barrier with a punch of the cap when the punch is pushed through the procedural barrier with by way of articulating the cap.

7. The portal-establishing device of claim 6, wherein the portal extends from the cap along a centerline of the punch but opposite thereof such that the portal barrier is aligned with the breach in the procedural barrier when the procedural barrier is breached by the punch.

8. The portal-establishing device of claim 1, wherein the portal-establishing device is sterile and the procedural barrier is a sterile drape.

9. A method of using a portal-establishing device, comprising:
breaching a procedural barrier between a single-use medical device on a clinician-facing side of the procedural barrier and a second medical device on a patient-facing side of the procedural barrier with a breaching mechanism of the portal-establishing device; and
placing a portal over a breach in the procedural barrier, thereby enabling one or more functional connections between the single-use medical device and the second medical device through the portal of the portal-establishing device.

10. The method of claim 9, wherein the breaching mechanism comprises a cap coupled to a rigid band by a hinge pin, further comprising:
closing the cap over a protruding portion of the second medical device; and
breaching the procedural barrier between the cap and the protruding portion of the second medical device with a cutting edge of the cap while closing the cap over the protruding portion of the second medical device.

11. The method of claim 9, wherein the breaching mechanism comprises a cap coupled to an extension arm by a hinge pin, further comprising:
articulating the extension arm over a protruding portion of the second medical device; and
breaching the procedural barrier between the cap and the protruding portion of the second medical device by pushing a punch of the extension arm through the procedural barrier while articulating the extension arm.

12. The method of claim 9, wherein the breaching mechanism comprises a cap coupled to a rigid band by a hinge pin, further comprising:
articulating the cap over a protruding portion of the second medical device; and
breaching the procedural barrier between the cap and the protruding portion of the second medical device by pushing a punch of the cap through the procedural barrier while articulating the cap.

13. The method of claim 9, wherein the portal-establishing device is sterile and the procedural barrier is a sterile drape.
